(19) United States
(12) Patent Application Publication
(10) Pub. No.: US 2026/0309609 A1
(43) Pub. Date: Oct. 8, 2026

(54) METHOD FOR PARTICLE SIZE ANALYSIS OF BED MATERIAL SEDIMENT AND RELATED DEVICE

(71) Applicant: CHINA THREE GORGES UNIVERSITY, Yichang City (CN)

(72) Inventors: Changwen LI, Yichang City (CN); Lingqi TAN, Yichang City (CN); Yifei ZHENG, Yichang City (CN); Jinglan LIU, Yichang City (CN); Chenxue GONG, Yichang City (CN); Yi YUAN, Yichang City (CN); Haoxiong ZHAN, Yichang City (CN); Ye HUANG, Yichang City (CN); Yichen YU, Yichang City (CN); Tao XU, Yichang City (CN); Xiaolei LV, Yichang City (CN)

(21) Appl. No.: 19/421,927

(22) Filed: Dec. 16, 2025

(30) Foreign Application Priority Data
Apr. 7, 2025 (CN) 202510430773.1

(51) Int. Cl. G01N 15/0205; G01N 1/10; G01N 1/38; G01N 1/40; G01N 15/00; G01N 15/02; G01N 15/04; G01N 15/075; G01N 33/24

(52) U.S. Cl. CPC G01N 15/0205; G01N 1/10; G01N 1/38; G01N 1/4044; G01N 15/04; G01N 15/075; G01N 33/24; G01N 2001/1025; G01N 2015/0053; G01N 2015/0283; G01N 2015/0288

(57) ABSTRACT

Provided is a method for particle size analysis of bed material sediment and a related device. The method includes: firstly, obtaining an intensity of light through a preset plane at each of a plurality of moments during a settling process of a bed material sediment sample; and bed material sediment with different particle sizes in the bed material sediment sample settles to the preset plane at different settling rates; for each particle size, calculating a percentage by weight of sediment finer than the particle size based on the intensity of light through the preset plane at each moment; and obtaining a particle size distribution and a particle size distribution curve based on each particle size and the percentage by weight of sediment finer than the particle size. Moreover, a vertical average particle size distribution, a cross-sectional average particle size distribution, and a cross-sectional average particle size can be further calculated.

### CROSS REFERENCE TO RELATED APPLICATION

This patent application claims the benefit and priority of Chinese Patent Application No. 2025104307731, filed with the China National Intellectual Property Administration on Apr. 7, 2025, the disclosure of which is incorporated by reference herein in its entirety as part of the present application.

### TECHNICAL FIELD

The present application relates to the technical field of hydraulic engineering and sediment measurement, and in particular, to a method for particle size analysis of bed material sediment and a related device.

### BACKGROUND

In recent decades, sediment transport in river source areas of many places, such as the Qinghai-Tibet Plateau, the Yellow River, and the Tarim River in Xinjiang, has shown an increasing trend. Rivers carry varying amounts of sediment, which silt up the rivers, raising the riverbeds year by year. Consequently, the rivers are prone to flooding and meandering, posing significant challenges for river regulation. Due to the relatively low river velocity in these areas, sediment deposits and becomes the bed material. The sediment in a reservoir area is mostly bed material sediment. Long-term deposition of the sediment leads to siltation of the reservoir, shortens the lifespan of the project, and reduces the flood control, irrigation, and power generation capacities of the project. The sediment also exacerbates the wear and tear of hydraulic machinery and structures, and increases maintenance costs and project costs, etc. However, the sediment has beneficial aspects: coarse particles serve as excellent construction materials, while fine sediments can be used for irrigation to improve soil, transforming saline-alkali wasteland into fertile farmland. Pumping water and desilting can strengthen embankments, thereby enhancing flood resistance and the like. To harness advantages and mitigate disadvantages, it is essential to conduct sediment sampling, perform sediment particle size analysis, and organize the data. This provides a reliable basis for watershed development and national economic construction, addressing key technical shortcomings in reducing siltation and discharging sediment in the reservoir area of the Qinghai-Tibet Plateau.

The main purpose of bed material sediment (i.e., bed material) sampling is to conduct particle size analysis of the bed material. This further aids in analyzing the cross-sectional distributions and variations of the suspended load concentration and the basic bed load transport rate, providing fundamental data for studying riverbed erosion and deposition changes as well as roughness. However, current methods for particle size analysis of bed material sediment suffer from inaccurate.

### SUMMARY

An objective of the present application is to provide a method for particle size analysis of bed material sediment and a related device that can accurately complete the particle size analysis of the bed material sediment.

To achieve the above objective, the present application provides the following technical solutions.

In a first aspect, the present application provides a method for particle size analysis of bed material sediment, including:
obtaining an intensity of light through a preset plane at each of a plurality of moments during a settling process of a bed material sediment sample, where the preset plane is any horizontal plane other than a highest horizontal plane and a lowest horizontal plane in a sample container holding an evenly mixed bed material sediment suspension; the evenly mixed bed material sediment suspension is obtained by evenly mixing the bed material sediment sample and distilled water or by evenly mixing the bed material sediment sample and salt-free water; and bed material sediment with different particle sizes in the bed material sediment sample settles to the preset plane at different settling rates;
for each of the particle sizes, calculating a percentage by weight of sediment finer than the particle size based on the intensity of light through the preset plane at each of the plurality of moments, where the percentage by weight of sediment finer than the particle size is a ratio of a sediment concentration of particle sizes smaller than the particle size to a total sediment concentration; and
obtaining a particle size distribution and a particle size distribution curve based on each of the particle sizes and the percentage by weight of sediment finer than the particle size.

In a second aspect, the present application provides a device for particle size analysis of bed material sediment, including:
a light intensity obtaining module configured to obtain an intensity of light through a preset plane at each of a plurality of moments during a settling process of a bed material sediment sample, where the preset plane is any horizontal plane other than a highest horizontal plane and a lowest horizontal plane in a sample container holding an evenly mixed bed material sediment suspension; the evenly mixed bed material sediment suspension is obtained by evenly mixing the bed material sediment sample and distilled water or by evenly mixing the bed material sediment sample and salt-free water; and bed material sediment with different particle sizes in the bed material sediment sample settles to the preset plane at different settling rates;
a percent calculation module configured to, for each of the particle sizes, calculate a percentage by weight of sediment finer than the particle size based on the intensity of light through the preset plane at each of the plurality of moments, where the percentage by weight of sediment finer than the particle size is a ratio of a sediment concentration of particle sizes smaller than the particle size to a total sediment concentration; and
a particle size analysis module configured to obtain a particle size distribution and a particle size distribution curve based on each of the particle sizes and the percentage by weight of sediment finer than the particle size.

In a third aspect, the present application provides a computer device including a memory, a processor, and a computer program stored on the memory and runnable on the processor, where the processor is configured to execute the computer program to implement the method for particle size analysis of bed material sediment described above.

In a fourth aspect, the present application provides a computer-readable storage medium, having a computer program stored therein which, when executed by a processor, causes the computer program to implement the method for particle size analysis of bed material sediment described above to be implemented.

In a fifth aspect, the present application provides a computer program product including a computer program which, when executed by a processor, causes the computer program to implement the method for particle size analysis of bed material sediment described above to be implemented.

According to specific embodiments provided in the present application, the present application has following technical effects.

The present application provides a method for particle size analysis of bed material sediment and a related device. An optical analysis method is adopted for particle size analysis of bed material sediment. The method specifically includes: firstly, obtaining an intensity of light through a preset plane at each of a plurality of moments during a settling process of a bed material sediment sample, where the preset plane is any horizontal plane other than a highest horizontal plane and a lowest horizontal plane in a sample container holding an evenly mixed bed material sediment suspension; and bed material sediment with different particle sizes in the bed material sediment sample settles to the preset plane at different settling rates; for each particle size, calculating a percentage by weight of sediment finer than the particle size based on the intensity of light through the preset plane at each moment; and obtaining a particle size distribution and a particle size distribution curve based on each particle size and the percentage by weight of sediment finer than the particle size. The optical analysis method is introduced in the present application to perform the particle size analysis of the bed material sediment with the intensity of light through the preset plane at each of the plurality of moments during the settling process of the bed material sediment sample. The particle size distribution and the particle size distribution curve are then calculated so that the particle size analysis of the bed material sediment can be completed accurately.

### BRIEF DESCRIPTION OF THE DRAWINGS

To describe the technical solutions in the embodiments of the present application or in the related art more clearly, the following briefly describes the accompanying drawings required for describing the embodiments or the related art. Apparently, the accompanying drawings in the following description show some embodiments of the present application, and a person of ordinary skill in the art may still derive other accompanying drawings from these accompanying drawings without creative efforts.

FIG. 1 is a diagram showing an environment in which a method for particle size analysis of bed material sediment is applied provided in Embodiment 1 of the present application;

FIG. 2 is a flowchart of a method for particle size analysis of bed material sediment provided in Embodiment 1 of the present application;

FIG. 3 is a schematic diagram showing a particle size analysis result of a bed material in Tarilega reservoir area 2 provided in Embodiment 1 of the present application;

FIG. 4 is a schematic diagram of functional modules of a device for particle size analysis of bed material sediment provided in Embodiment 2 of the present application; and

FIG. 5 is a schematic structural diagram of a computer device provided in Embodiment 3 of the present application.

### DETAILED DESCRIPTION OF THE EMBODIMENTS

The technical solutions in the embodiments of the present application are clearly and completely described below with reference to the drawings in the embodiments of the present application. Apparently, the described embodiments are only some rather than all of the embodiments of the present application. All other embodiments derived from the embodiments in the present application by a person of ordinary skill in the art without creative efforts should fall within the protection scope of the present application.

### Embodiment 1

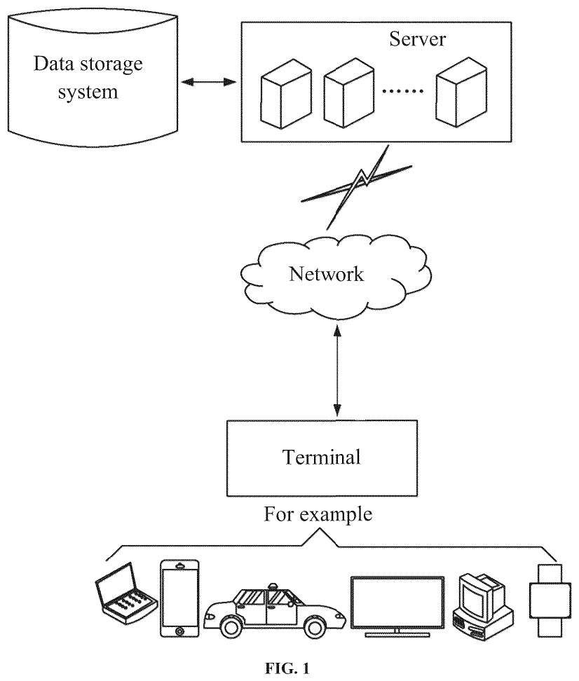

A method for particle size analysis of bed material sediment provided by an embodiment of the present application may be applied to the environment as shown in FIG. 1. A terminal communicates with a server over a network. A data storage system may store data needing to be processed by the server. The data storage system may be arranged separately, or integrated with the server, or disposed on cloud or other servers. The terminal may send an analysis request to be processed to the server. After the server receives the analysis request to be processed, for the analysis request to be processed, the server obtains an intensity of light through a preset plane at each of a plurality of moments during a settling process of a bed material sediment sample, calculates, for each particle size, a percentage by weight of sediment finer than the particle size based on the intensity of light through the preset plane at each moment, and obtains a particle size distribution and a particle size distribution curve based on each particle size and the percentage by weight of sediment finer than the particle size. The server may feed back to the terminal the analysis results particle size distribution and particle size distribution curve obtained against the analysis request.

Furthermore, in some embodiments, the method for particle size analysis of bed material sediment may also be implemented by the server or the terminal alone. For example, the analysis request to be processed may be directly processed by the terminal. Alternatively, the analysis request to be processed may be acquired from the data storage system and then processed by the server.

The terminal may be, but not limited to, various desktop computers, laptop computers, smartphones, tablet computers, Internet-of-Things devices, and portable wearable devices. The Internet-of-Things devices may be smart speakers, smart televisions, smart air conditioners, smart vehicular devices, etc. The portable wearable devices may be smart watches, smart bracelets, headset devices, etc. The server can be implemented by a standalone server or a server cluster made up of a plurality of servers, or may be a cloud server.

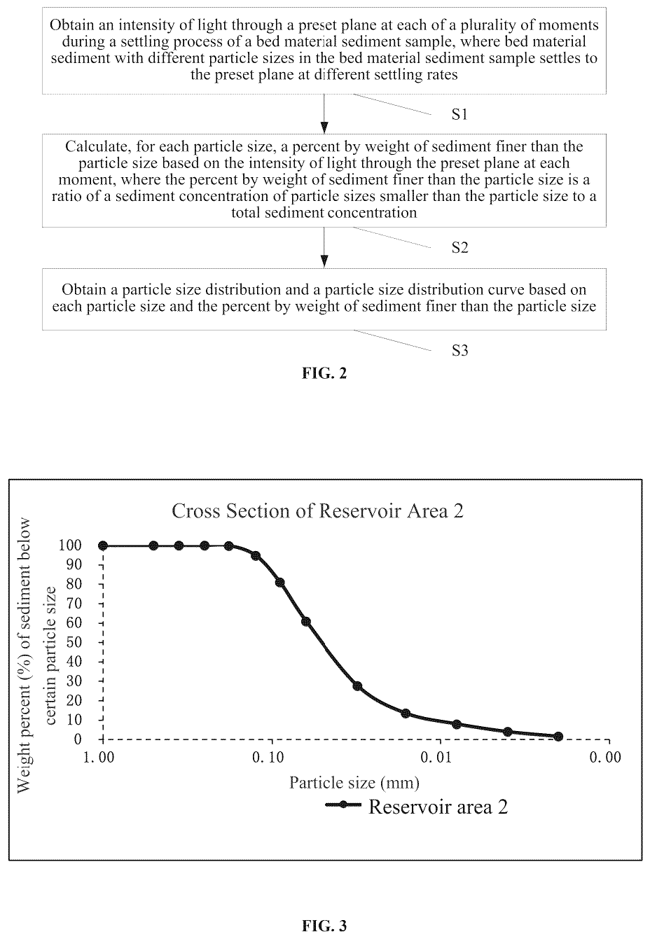

In an exemplary embodiment, as shown in FIG. 2, provided is a method for particle size analysis of bed material sediment. The method is performed by a computer device, specifically performed separately by a computer device such as a terminal or a server, or performed jointly by a terminal and a server. In an embodiment of the present application, the method for particle size analysis of bed material sediment is applied to the server in FIG. 1 for example, and includes the following steps.

In step S1, an intensity of light through a preset plane at each of a plurality of moments during a settling process of a bed material sediment sample is obtained, where the preset plane is any horizontal plane other than a highest horizontal plane and a lowest horizontal plane in a sample container holding an evenly mixed bed material sediment suspension; the evenly mixed bed material sediment suspension is obtained by evenly mixing the bed material sediment sample and distilled water or by evenly mixing the bed material sediment sample and salt-free water; and bed material sediment with different particle sizes in the bed material sediment sample settles to the preset plane at different settling rates.

In step S2, for each particle size, a percentage by weight of sediment finer than the particle size is calculated based on the intensity of light through the preset plane at each of the plurality of moments, where the percentage by weight of sediment finer than the particle size is a ratio of a sediment concentration of particle sizes smaller than the particle size to a total sediment concentration.

In step S3, a particle size distribution and a particle size distribution curve are obtained based on each particle size and the percentage by weight of sediment finer than the particle size.

By carrying out the aforesaid steps S1 to S3, this embodiment, using the optical analysis method, allows for more accurate particle size analysis of the bed material sediment by obtaining the intensity of light through the preset plane at each of the plurality of moments during the settling process of the bed material sediment sample, calculating the percentage by weight of sediment finer than each particle size based on the intensity of light through the preset plane at each moment, and obtaining the particle size distribution and a particle size distribution curve based on each particle size and the percentage by weight of sediment finer than the particle size.

In this embodiment, the sample container further holds a deflocculant to avoid agglomeration and settling of the bed material sediment sample in the sample container.

In this embodiment, a formula for calculating the percentage by weight of sediment finer than the particle size is as follows:

$${P}_{i} = \frac{{\sum }_{i = i}^{n - 1} \frac{2.3 {D}_{i , i + 1}}{kL} ( lg \frac{{I}_{0}}{{I}_{{t}_{i}}} - lg \frac{{I}_{0}}{{I}_{{t}_{i + 1}}} )}{{\sum }_{i = 1}^{n - 1} \frac{2.3 {D}_{i , i + 1}}{kL} ( lg \frac{{I}_{0}}{{I}_{{t}_{i}}} - lg \frac{{I}_{0}}{{I}_{{t}_{i + 1}}} )}$$

where Pᵢ represents a percentage by weight of sediment finer than an i-th particle size; n represents a number of particle sizes; D_{i, i+1} represents a particle size between the i-th particle size and an (i+1)-th particle size; k represents an extinction coefficient; L represents a thickness of the evenly mixed bed material sediment suspension; I₀ represents a light intensity at a 0-th moment, i.e., an intensity of light through distilled water or salt-free water; Iₜ_{ᵢ} represents a light intensity at a moment tᵢ when the bed material sediment of the i-th particle size settles to the preset plane; and Iₜ_{ᵢ₊₁} represents a light intensity at a moment tᵢ₊₁ when the bed material sediment of the (i+1)-th particle size settles to the preset plane.

In this embodiment, suspended load measurement or bed load measurement may be further completed. The suspended load measurement is applied in hydraulic engineering, river regulation, and soil and water conservation. The bed load measurement is used for studies on reservoir sedimentation and riverbed evolution, thereby facilitating subsequent use. When the method for particle size analysis of bed material sediment is applied to suspended load measurement or bed load measurement, the bed material sediment sample is a sample obtained after performing water sample treatment on a bed material sediment water sample; and the bed material sediment water sample is a bed material sediment water sample of each layer obtained by performing stratified sampling at each bed material sampling vertical at each measurement run.

The water sample treatment includes filtering and drying the bed material sediment water sample to obtain the bed material sediment sample.

The stratified sampling refers to sampling at different bed material sampling vertical depths. During stratified sampling, a formula for calculating a sampling volume of the bed material sediment water sample is as follows:

$$V = \frac{{W}_{min}}{C}$$

where V represents the sampling volume of the bed material sediment water sample; Wₘᵢₙ represents a minimum sediment weight; and C represents a sediment concentration.

The method for particle size analysis of bed material sediment in this embodiment further includes the step of calculating a vertical average particle size distribution at each bed material sampling vertical by the following formula:

$${\overline{P}}_{v} ( d ) = \frac{1}{H} {\sum }_{k = 1}^{m} {P}_{k} ( d ) \cdot Δ {h}_{k}$$

where P̅ᵥ̅(d) represents the vertical average particle size distribution; d represents a particle size; H represents a vertical depth; m represents a number of layers for stratified sampling; Pₖ(d) represents a particle size distribution at a k-th layer, i.e., the particle size distribution obtained by treating the bed material sediment sample collected at the k layer using steps S1 to S3; and Δhₖ represents a sampling thickness of the k-th layer.

The method for particle size analysis of bed material sediment in this embodiment further includes the step of calculating a cross-sectional average particle size distribution and a cross-sectional average particle size of a cross section composed of all bed material sampling verticals at each measurement run.

A formula for calculating the cross-sectional average particle size distribution is as follows:

$$\overline{{P}_{s}} ( d ) = \frac{{\sum }_{i = 1}^{X} {W}_{i} \cdot \overline{{P}_{{v}_{l}}} ( d )}{{\sum }_{i = 1}^{X} {W}_{i}}$$

where P̅ₛ(d) represents the cross-sectional average particle size distribution; X represents a number of bed material sampling verticals; Wi represents a sediment weight per unit width at an i-th bed material sampling vertical; and
P̅ᵥ̅_̅{̅ₜ̅}̅(d) represents the vertical average particle size distribution at the i-th bed material sampling vertical.

$${W}_{i} = {\int }_{0}^{D} {ρ}_{S} \cdot {A}_{i} ( d ) \cdot d ( d )$$

where D represents a maximum particle size; ρₛ represents a bed material sediment density; and Aᵢ(d) represents a particle size distribution function for the i-th bed material sampling vertical.

A formula for calculating the cross-sectional average particle size is as follows:

$${D}_{a v g} = exp ( {\int }_{{d}_{min}}^{{d}_{max}} lnd \cdot d \overline{{P}_{s}} ( d ) ) ;$$

where D_{avg} represents the cross-sectional average particle size; dₘₐₓ represents a maximum particle size value; and dₘᵢₙ represents a minimum maximum particle size value.

This embodiment may provide a method for particle size analysis of a bed material sediment sample obtained by automatically sampling the bed material sediment in a reservoir, a river, a lake, or the like, i.e., provides a method for particle size analysis of bed material sediment that enables scientific, systematic, efficient, and accurate particle size analysis of the bed material sediment.

Hereinafter, taking its use in suspended load measurement or bed load measurement as an example, the method for particle size analysis of bed material sediment in this embodiment is described in detail. The method for particle size analysis of bed material sediment includes the following steps.

In step 1, bed material sediment is sampled.

For particle size analysis of the bed material sediment, sediment is sampled. During each measurement run for particle size analysis in suspended load measurement or bed load measurement, the bed material sediment is simultaneously collected. The number of measurement runs can be determined based on user requirements.

At each measurement run, stratified sampling is conducted at each bed material sampling vertical, where the locations of the bed material sampling verticals coincide with those for the suspended load or bed load measurement. The number of bed material sampling verticals is determined as follows: Based on the river width and sediment distribution characteristics, the bed material sampling verticals are deployed according to the requirements stipulated in the “Regulation for Particle Size Analysis of River Sediment” (SL42-2010), covering the main flow zone, shoals, and transition areas.

In step 2, a sampling amount of the bed material sediment sample is calculated and the bed material sediment sample is treated.

The desired sampling amount is calculated and the sediment sample is treated.

(1) The sampling amount of the bed material sediment sample is calculated.

In this embodiment, reference is made to the “Regulation for Particle Size Analysis of River Sediment” (SL 42-2010). For bed material sediment, it is generally required that the dry weight of each bed material sediment sample is not less than 0.1 g to 0.5 g. During sampling, it shall be ensured that the bed material sediment sample is representative of the overall characteristics of the bed material, thereby avoiding data deviation caused by an insufficient amount of the bed material sediment sample. In such cases, the sampling volume V of the corresponding bed material sediment water sample can be calculated based on the sediment concentration C (kg/m³ or g/L) and the minimum sediment weight:

$$V = \frac{{W}_{min}}{C}$$

The sampling volume of the bed material sediment water sample is generally 0.5 L to 5 L.

(2) The sediment sample is treated.

When the water analysis method is utilized to analyze the bed material sediment sample, moist bed material sediment samples must be used. Except for coarse sediment and pebbles analyzed entirely by sieving, the use of dry sediment for analysis is prohibited. For this purpose, the water sample is treated by the displacement method. After treatment, the water sample can be retained for particle size analysis. Specifically, the bed material sediment water sample is treated using the filtration and drying methods, and specific steps are as follows.
1) After sampling is completed, the bed material sediment water sample is filtered using standard filter paper or a filter membrane to separate the sediment from the water. The pore size of the filter paper is typically 0.45 micrometers. After the filtration is completed, the sediment sample is placed in a sealed container.
2) Drying: the sediment sample is placed in a drying oven and dried at 105° C. until a constant weight is achieved, ensuring all moisture is evaporated.
3) Weighing: After the drying is completed, the weight m_{d} of the dried sediment is measured.
4) Calculation: The dry bulk density ρ_{d} of the sediment is calculated according to the following formula:

$${ρ}_{d} = \frac{{m}_{d}}{V} ;$$

where ρ_{d} represents the dry bulk density of the sediment (in units of g/cm³ or kg/m³); m_{d} represents the mass of the sediment in a completely dry state (in units of g or kg); and V represents the volume of the sediment (in units of cm³ or m³).

During the sedimentation and concentration of the water sample for particle size analysis, no chemical can be used to accelerate the sedimentation. The water analysis method must use distilled water or salt-free water produced by ion exchange resin. To prevent the bed material sediment sample from agglomerating and settling during analysis, a deflocculant may be added to the concentrated water sample (i.e., distilled water or salt-free water). Generally, ammonium hydroxide at the concentration of 25% is used for deflocculation. Other chemicals with better deflocculation effect, such as sodium hexametaphosphate and water glass, may alternatively be added.

The collected bed material sediment water sample is left to stand for 1 d. If flocculation is observed at the top of the settled sediment, it indicates the presence of water-soluble salts causing the sediment to agglomerate and settle. In this case, the following methods may be applied to address this issue.
1) Flushing Method: the water sample is poured into a beaker and heated to boiling. After the water sample is left to stand for sedimentation, a portion of the clear water is removed. Then, the water sample is diluted with hot distilled water and left to settle again, and the clear water is removed. This process is repeated until the water-soluble salts are eliminated.
2) Filtration Method: a hardened filter paper is attached to a funnel. The sediment sample is poured into the funnel, and then filtered by adding hot distilled water thereto. During filtration, the liquid level in the funnel is maintained approximately 5 mm above the sediment sample until the water-soluble salts have been completely filtered out.

In step 3, particle size analysis of the sediment is performed using the optical analysis method under the water analysis method to calculate a particle size distribution and a particle size distribution curve.

Based on the particle composition of the bed material sediment, the particle size distribution is analyzed and the particle size distribution curve is plotted. The abscissas of the particle size distribution curve are logarithmic coordinates, representing the particle sizes of the sediment, while the ordinates are probability grid coordinates, representing the percentages by weight of sediment finer than a given particle size. This step includes the measurement of extinction and the calculation of the particle size distribution.

(1) Light intensity attenuation is dynamically monitored and a model is constructed.

The bed material sediment sample is put into distilled water or salt-free water and stirred evenly to form an evenly mixed bed material sediment suspension. Based on a photoelectric particle analyzer, the variation in light intensity during the settling process of the bed material sediment sample is monitored in real time, and a quantitative relationship model between the extinction value and particle characteristics is constructed:

$$E = lg \frac{{I}_{0}}{I}$$

where E represents an extinction value; and I₀ and I represent intensities of light after passing through pure water (i.e., distilled water or salt-free water) and turbid water (i.e., the evenly mixed bed material sediment suspension, in particular distilled water or salt-free water containing the bed material sediment sample), respectively.

(2) The extinction value and particle parameters are dynamically correlated.

A mathematical model between E and a sediment concentration Cₛ, a particle size D, and an optical path length L is as follows:

$$lg \frac{{I}_{0}}{I} = \frac{1}{2.3} \frac{{kC}_{s} L}{D} ;$$

where k represents an extinction coefficient, which is related to the sediment particle geometry; Cₛ represents a sediment concentration of turbid water; L represents a thickness of the turbid water; and D represents a sediment particle size.

(3) Phased dynamic modeling of the settling process is carried out.
1) After the water sample is mixed evenly, at moment t₁, the sediment of the first particle size D₁ just settles to the incident light (i.e., the preset plane), and at this point, the extinction value is calculated by the following formula:

$$lg \frac{{I}_{0}}{{I}_{{t}_{1}}} = lg \frac{{I}_{0}}{{I}_{1}} + lg \frac{{I}_{0}}{{I}_{2}} + ⋯ + lg \frac{{I}_{0}}{{I}_{i}} ⋯ + lg \frac{{I}_{0}}{{I}_{n}} = {\sum }_{i = 1}^{n} lg \frac{{I}_{0}}{{I}_{i}} ;$$

where Iₜ_{₁} represents a light intensity at moment t₁ when the bed material sediment of the first particle size settles to the preset plane; I₁, I₂, . . . , and Iᵢ represent light intensities after extinction simply caused by the sediment with particle sizes D₁, D₂, . . . , Dᵢ; D₂ represents a second particle size, and Dᵢ represents an i-th particle size; Iₙ represents a light intensity after extinction simply caused by the sediment of a minimum n-th particle sizeDᵢ; and

$$lg \frac{{I}_{0}}{{I}_{1}} , lg \frac{{I}_{0}}{{I}_{2}} , lg \frac{{I}_{0}}{{I}_{i}}$$

and

$$lg \frac{{I}_{0}}{{I}_{n}}$$

represent extinction values separately caused by the sediment of various particle sizes.
2) At moment t₂, the sediment above the particle size D₂ settles below the incident light and the sediment of the particle size D₂ just settles to the incident light. At this point, the extinction value is calculated by the following formula:

$$lg \frac{{I}_{0}}{{I}_{{t}_{2}}} = lg \frac{{I}_{0}}{{I}_{2}} + ⋯ + lg \frac{{I}_{0}}{{I}_{i}} ⋯ + lg \frac{{I}_{0}}{{I}_{n}} = {\sum }_{i = 1}^{n} lg \frac{{I}_{0}}{{I}_{i}} ;$$

where Iₜ_{₂} represents a light intensity at moment t₂ when the bed material sediment of the second particle size settles to the preset plane.
3) The light intensity after extinction caused by the sediment with the particle sizes D₁~D₂ is calculated by the following formula:

$$lg \frac{{I}_{0}}{{I}_{1 , 2}} = lg \frac{{I}_{0}}{{I}_{{t}_{1}}} - lg \frac{{I}_{0}}{{I}_{{t}_{2}}} ;$$

$$lg \frac{{I}_{0}}{{I}_{{t}_{1}}} - lg \frac{{I}_{0}}{{I}_{{t}_{2}}} = \frac{1}{2.3} \frac{{kC}_{S 1 , 2} L}{{D}_{1 , 2}} ,$$

where

$$lg \frac{{I}_{0}}{{I}_{1 , 2}}$$

represents the extinction value of the sediment with the particle sizes D₁-D₂; I_{1,2} represents the light intensity after extinction caused by the sediment with the particle sizes D₁-D₂; C_{s1,2} represents the sediment concentration with the particle sizes D₁-D₂; and D_{1,2} represents the particle size of the sediment with the particle sizes D₁-D₂.

(4) Recursive calculation of a fractional sediment concentration and correction of the extinction coefficient are carried out.
1) The sediment concentration with the particle sizes D₁-D₂ is calculated by the following formula:

$${C}_{s 1 , 2} = \frac{2.3 {D}_{1 , 2}}{kL} ( lg \frac{{I}_{0}}{{I}_{{t}_{1}}} - lg \frac{{I}_{0}}{{I}_{{t}_{2}}} )$$

2) The sediment concentration with the particle sizes D₂-D₃ is calculated by the following formula:

$${C}_{s 2 , 3} = \frac{2.3 {D}_{2 , 3}}{kL} ( lg \frac{{I}_{0}}{{I}_{{t}_{2}}} - lg \frac{{I}_{0}}{{I}_{{t}_{3}}} ) ;$$

where C_{s2,3} represents the sediment concentration with the particle sizes D₂-D₃; D_{2,3} represents the particle size of the sediment with the particle sizes D₂-D₃; and Iₜ_{₃} represents a light intensity at moment t₃ when the bed material sediment of a third particle size settles to the preset plane.
3) A total sediment concentration in the suspension water sample is equal to a sum of the sediment concentrations of various particle sizes, and calculated by the following formula:

$${C}_{s} = {C}_{s 1 , 2} + {C}_{s 2 , 3} + ⋯ + {C}_{si , i + 1} + ⋯ + {C}_{sn - 1 , n}$$

where C_{si,i+1} represents the sediment concentration with the particle sizes Dᵢ to Dᵢ₊₁; Dᵢ₊₁ represents an (i+1)-th particle size; C_{sn-1,n} represents the sediment concentration with the particle sizes Dₙ₋₁ to Dₙ; and Dₙ₋₁ represents an (n−1)-th particle size.

4) The percentage by weight of sediment finer than a certain particle size in the evenly mixed suspension may be derived from a ratio of the sediment concentration with various particle sizes smaller than the certain particle size to a total sediment concentration:

$${P}_{i} = \frac{{C}_{si , i + 1} + {C}_{si + 1 , i + 2} + ⋯ + {C}_{sn - 1 , n}}{{C}_{s 1 , 2} + {C}_{s 2 , 3} + ⋯ + {C}_{si , i + 1} + {C}_{si + 1 , i + 2} ⋯ + {C}_{sn - 1 , n}}$$

$$= \frac{{\sum }_{i = i}^{n - 1} {C}_{si , i + 1}}{{\sum }_{i = 1}^{n - 1} {C}_{si , i + 1}}$$

$$= \frac{{\sum }_{i = i}^{n - 1} \frac{2.3 {D}_{i , i + 1}}{kL} ( lg \frac{{I}_{0}}{{I}_{{t}_{i}}} - lg \frac{{I}_{0}}{{I}_{{t}_{i + 1}}} )}{{\sum }_{i = 1}^{n - 1} \frac{2.3 {D}_{i , i + 1}}{kL} ( lg \frac{{I}_{0}}{{I}_{{t}_{i}}} - lg \frac{{I}_{0}}{{I}_{{t}_{i + 1}}} )}$$

where Pᵢ represents a percentage by weight of sediment finer than an i-th particle size; n represents a number of particle sizes; D_{i, i+1} represents a particle size between the i-th particle size and an (i+1)-th particle size; k represents an extinction coefficient; L represents a thickness of the evenly mixed bed material sediment suspension; I₀ represents a light intensity at a 0-th moment; Iₜ_{ᵢ} represents a light intensity at a moment tᵢ when the bed material sediment of the i-th particle size settles to the preset plane; and Iₜ_{ᵢ₊₁} represents a light intensity at a moment tᵢ₊₁ when the bed material sediment of the (i+1)-th particle size settles to the preset plane.

Due to the known particle sizes D₁ to Dₙ and the light intensity at each moment, the moment when the sediment of each particle size settles to the preset plane can be determined by an existing sediment settling formula so that Iₜ_{ᵢ} and Iₜ_{ᵢ₊₁} can be determined. Thus, the weight percent of the sediment of this particle size can be calculated. It should be noted that D₁ to Dₙ are a plurality of particle sizes selected according to requirements. There will be other particle sizes between two adjacent particle sizes, and therefore, there exists D_{i, i+1}, which refers to the particle size between the i-th particle size and the (i+1)-th particle size.

In step 4, a vertical average particle size distribution, a cross-sectional average particle size distribution, and a cross-sectional average particle size are calculated.

The particle size distributions obtained by the optical analysis method are collated, whereby the vertical average particle size distribution, the cross-sectional average particle size distribution, and the cross-sectional average particle size of the bed material sediment may be derived, and the cross-sectional average particle size distribution curve may further be plotted.

(1) An integral calculation model for the vertical average particle size distribution is established.

A discretized integral model is established for the bed material sediment sample collected at the bed material sediment sampling vertical using the depth-integration method. If the vertical depth H is divided into m equally spaced layers (i.e., sampling layers) and the particle size distribution of the k-th layer is P_{K}(d), the vertical average particle size distribution P̅ₖ̅(d) is as follows:

$${\overline{P}}_{v} ( d ) = \frac{1}{H} {\sum }_{k = 1}^{m} {P}_{k} ( d ) \cdot Δ {h}_{k}$$

where d represents the particle size; and Δhₖ represents the sampling thickness of the k-th layer and meets

$${\sum }_{k = 1}^{m} Δ {h}_{k} = H .$$

(2) Vector superposition calculation of cross-sectional average particle size distributions is carried out.

For the calculation of the cross-sectional average particle size distribution, particle size analysis is performed on samples collected by the full-cross section mixing method, and the results can be used as the cross-sectional average particle size distribution curve. It is defined that the cross section is composed of n bed material sampling verticals forming a spatial distribution matrix, and a vector superposition model based on sediment weight weighting is established:

$$\overline{{P}_{s}} ( d ) = \frac{{\sum }_{i = 1}^{x} {W}_{i} \cdot \overline{{P}_{{v}_{ι}}} ( d )}{{\sum }_{i = 1}^{X} {W}_{i}}$$

where P̅ₛ(d) represents the cross-sectional average particle size distribution; X represents the number of bed material sampling verticals; and Wᵢ represents the sediment weight per unit width at the i-th bed material sampling vertical, which is calculated by the following formula:

$${W}_{i} = {\int }_{0}^{D} {ρ}_{s} \cdot {A}_{i} ( d ) \cdot d ( d )$$

where D represents the maximum particle size; ρₛ represents the bed material sediment density; and Aᵢ(d) represents the particle size distribution function (kg/m³·mm⁻¹) for the i-th bed material sampling vertical.

(3) Fractal analytical calculation of the cross-sectional average particle size is carried out.
1) Logistic curve discretization is performed on P̅ₛ(d), and the particle size interval [dₘᵢₙ, dₘₐₓ] is divided into p geometrical progression groups:

$${d}_{j} = \sqrt{{d}_{j}^{L} \cdot {d}_{j}^{U}}$$

where dⱼ represents a representative value of a j-th particle size; and

$${d}_{j}^{L} and {d}_{j}^{U}$$

represent an upper limit and a lower limit of the j-th particle size, respectively.
2) A differential equation for a particle size distribution is established:

$$Δ {P}_{j} = \overline{{P}_{s}} ( {d}_{j}^{U} ) - \overline{{P}_{s}} ( {d}_{j}^{L} )$$

where ΔPⱼ represents a percent increase (%) in sediment weight corresponding to the j-th group of particle sizes, representing a cumulative probability variation of the cross-sectional average particle size distribution curve within a specific particle size interval;

$$\overline{{P}_{s}} ( {d}_{j}^{U} )$$

represents a value of a cross-sectional average particle size distribution function at an upper limit of the j-th group of particle sizes; and

$$\overline{{P}_{s}} ( {d}_{j}^{L} )$$

represents a value of the cross-sectional average particle size distribution function at a lower limit of the j-th group of particle sizes.

By this differential equation, the sediment weight proportions for the particle size groups are quantified with an algebraic difference

$$\overline{{P}_{s}} ( {d}_{j}^{U} ) - \overline{{P}_{s}} ( {d}_{j}^{L} )$$

of end point particle size distribution values, providing key weight parameters for subsequent weighted calculation of a geometric average particle size.
3) A fractal weighting matrix is constructed:

$${D}_{a v g} = exp ( \frac{{\sum }_{j = 1}^{p} Δ {P}_{j} \cdot ln {d}_{j}}{{\sum }_{j = 1}^{p} Δ {P}_{j}} )$$

where D_{avg} represents the cross-sectional average particle size.

When ΔPⱼ→0, the equation takes a continuous integral form:

$${D}_{a v g} = exp ( {\int }_{{d}_{min}}^{{d}_{max}} ln d \cdot d \overline{{P}_{s}} ( d ) )$$

A two-level integration system of vertical-cross section is established using

$$\overline{{P}_{v}} ( d ) = \frac{1}{H} {\sum }_{k = 1}^{m} {P}_{k} ( d ) \cdot Δ {h}_{k}$$

and

$$\overline{{P}_{s}} ( d ) = \frac{{Σ}_{i = 1}^{X} {W}_{i} \cdot \overline{{P}_{{v}_{l}}} ( d )}{{Σ}_{i = 1}^{X} {W}_{i}}$$

in this embodiment. The operability of practical engineering measurements is achieved through spatial discretization. By

$${d}_{j} = \sqrt{{d}_{j}^{L} \cdot {d}_{j}^{U}} , the$$

geometric progression grouping method is adopted to ensure computational stability over a wide particle size range. By

$${D}_{a v g} = exp ( {\int }_{{d}_{max}}^{{d}_{min}} ln d \cdot d \overline{{P}_{s}} ( d ) ) ,$$

the fractal geometry theory is innovatively introduced, whereby the particle size distribution characteristics of non-uniform sediment are accurately characterized via exponential weighted averaging. With the entire algorithm, a traceable mathematical model from microscopic particle size analysis to macroscopic cross-sectional parameters is established, and a closed-loop calculation chain is formed with weight transfer factors among the formulas.

This embodiment is employed for bed material sediment sampling and measurement at a reservoir. The reservoir exhibits a low water flow velocity and significant sediment siltation, with 96.8% bed material sediment, making it representative for particle size analysis of bed material sediment in the reservoir area. Through research and analysis, the advantages and positive effects of this embodiment are as follows:
(1) Scientificity and Systematicness of the Method for Particle Size Analysis of Bed Material Sediment By controlling the sampling amount of the sediment sample and applying the optical analysis method, this embodiment can rapidly and accurately obtain the particle size distribution. In the measurement for a reservoir, the particle size analysis results cover the range of 0.002 mm to 2 mm, providing a scientific basis for reservoir sedimentation control. Specifically, it offers reliable technical support for reservoir desilting, river regulation, and ecological environment protection.
(2) Accuracy of the Method for Particle Size Analysis of Bed Material Sediment The geometric progression grouping method is utilized to discretize the particle size interval, effectively resolving computational stability issues across large particle size ranges. The fractal geometry theory is introduced, and the particle size distribution characteristics of non-uniform sediment are quantified through exponential weighted averaging, enhancing the characterization capability for complex sediment structures. Simultaneously, the two-level integration system of vertical-cross-section, via spatial discretization modeling, seamlessly connects microscopic particle size analysis results (vertical average particle size distribution) to macroscopic cross-sectional parameters (cross-sectional average particle sizes), forming a full-chain mathematical model from data acquisition to engineering application.
(3) Efficiency of the Method for Particle Size Analysis of Bed Material Sediment The closed-loop calculation chain of the weight transfer factors in the algorithm design, along with phased dynamic modeling (e.g., phased extinction value calculation during the settling process), simplifies the complexity of practical operations and enhances executability in engineering scenarios. The entire process, from stratified sampling to bed material particle size calculation, strictly adheres to industry standards (e.g., SL42-2010), balancing scientific rigor with engineering implementation efficiency.

Taking the Tarilega Hydropower Station in 2024 as an example, for the arrangement of measurement runs and sampling operations for the particle size analysis of the bed material sediment, the specific implementation process involved interpreting satellite remote sensing images to establish a macroscopic watershed sediment distribution model. Unmanned vessels equipped with sampling devices and photoelectric particle analyzers were deployed simultaneously to conduct underwater dynamic sampling.

For the reservoir area, systematic sampling is conducted during the late dry season (April 18 to April 20). Following the requirements of the “Regulation for Particle Size Analysis of River Sediment” (SL42-2020), 21 characteristic cross sections are deployed within a 30 km study reach, using a change rate of channel curvature radius >15% as the control condition. Five bed material sediment sampling verticals are set up at each cross section for stratified sampling, including surficial sampling once, sampling once at 0.5 m depth (middle layer), and sampling once at 1 m depth (bottom layer), thereby obtaining a total of 18 samples per single cross section.

Hydrological parameters are recorded during sampling:flow velocity: 0.8-1.5 m/s; water temperature: 4.2-6.8° C.; and sediment concentration: 0.35-1.72 kg/m³. Sediment sample processing follows the following steps: first, the bed material particles are filtered; then, the bed material is dried in an oven; and finally, the dry bulk density of the sediment is determined using the oven-drying and weighing method. The dry bulk density data of the sediment is shown in Table 1 below.

TABLE 1
Dry Bulk Density Result Table for Tarilega

| Cross | | | | | Sampling | |
|---|---|---|---|---|---|---|
| Section | Sampling | Sampling | Sampling | Sampling | Volume | Weight |
| Name | Item | Position | Method | Bucket No. | (ml) | (g) |
| Dry bulk | Dry bulk | Surface | Stratified | Dry bulk | 250 | 352.2 |
| density | density | layer | sampling | density-surface | | |
| cross | | | | layer | | |
| section | Dry bulk | 0.5 m | Stratified | Dry bulk | 250 | 372.9 |
| | density | | sampling | density-0.5 | | |
| | Dry bulk | 1.0 m | Stratified | Dry bulk | 250 | 351.0 |
| | density | | sampling | density-1.0 | | |

Particle size analysis of the bed material sediment in the reservoir area is performed using the optical analysis method under the water analysis method. This includes monitoring of light intensity attenuation, correlation of the extinction value with particle parameters, phased modeling of the settling process, and recursive calculation of the size distribution-based sediment concentration. It also includes the calculation of the vertical and cross-sectional average particle size distributions, as well as the fractal analysis of the cross-sectional average particle size.

The particle size analysis results of the bed material for a typical cross section (e.g., #reservoir area 2 right cross section) are shown in Table 2 and FIG. 3. The results indicate that the median particle size D50 of the bed material is 0.056 m, which aligns with the deposition characteristics of mountainous rivers.

TABLE 2
Particle Size Analysis Result Table of Bed Material for Tarilega

| | | Areal Weight Percent (%) of Sediment | | | | | | | | |
|---|---|---|---|---|---|---|---|---|---|---|
| | | Finer Than Certain Particle Size | | | | | | | | |
| Measurement | Sampling | Particle Size Grade (mm) | | | | | | | | |
| Date | Position | 0.002 | 0.004 | 0.008 | 0.016 | 0.031 | 0.063 | 0.090 | 0.125 | 0.180 |
| 08.31 | Middle | 1.6 | 4.0 | 8.0 | 13.5 | 27.7 | 61.0 | 81.1 | 94.8 | 99.9 |

| | | | | | | Characteristic | | |
|---|---|---|---|---|---|---|---|---|
| | | | | | | Particle Size | | |
| | Areal Weight Percent (%) of Sediment | | | | | Median | Average | Maximum |
| | Finer Than Certain Particle Size | | | | | Particle | Particle | Particle |
| Measurement | Particle Size Grade (mm) | | | | | Size | Size | Size |
| Date | 0.250 | 0.355 | 0.500 | 1.00 | 2.00 | (mm) | (mm) | (mm) |
| 08.31 | 100 | | | | | 0.051 | 0.056 | 0.184 |

The present application further provides a scenario in which the aforementioned method for particle size analysis of bed material sediment is applied. Specifically, the method for particle size analysis of bed material sediment provided in this embodiment can be applied in a reservoir regulation scenario. The reservoir regulation scenario includes a particle size analysis process and a regulation process. The particle size analysis process is performed to analyze the particle sizes of the bed material sediment, and the regulation process is performed to implement reservoir regulation based on the particle size analysis results. The method for particle size analysis of bed material sediment provided in this embodiment belongs to the particle size analysis process.

### Embodiment 2

Based on the same inventive concept, an embodiment of the present application further provides a device for particle size analysis of bed material sediment that is configured to implement the aforesaid method for particle size analysis of bed material sediment. The implementation solutions to problems provided by the device are similar to those described in the aforesaid method. Thus, for specific definitions in one or more embodiments of the device for particle size analysis of bed material sediment, reference may be made to those provided above in the method for particle size analysis of bed material sediment, which will not be described here redundantly.

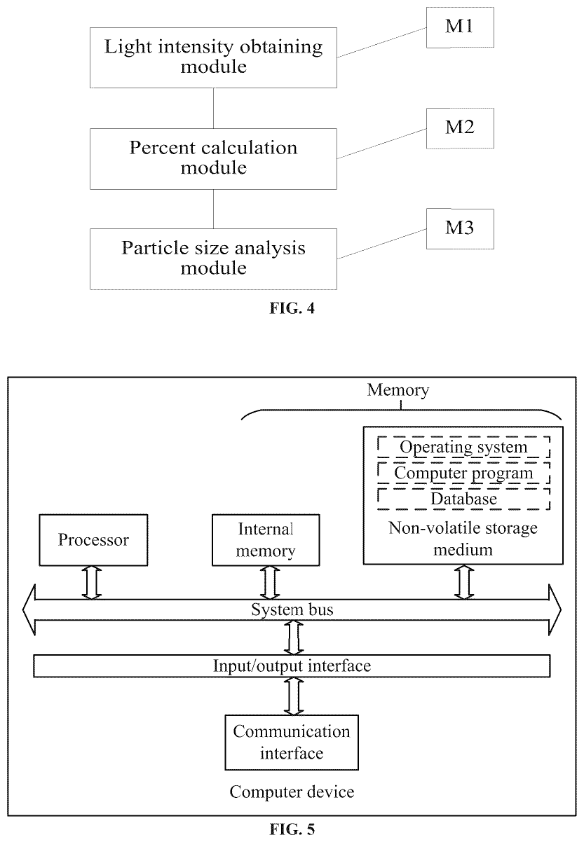

In an exemplary embodiment, as shown in FIG. 4, provided is a device for particle size analysis of bed material sediment, including:
a light intensity obtaining module M1 configured to obtain an intensity of light through a preset plane at each of a plurality of moments during a settling process of a bed material sediment sample, where the preset plane is any horizontal plane other than a highest horizontal plane and a lowest horizontal plane in a sample container holding an evenly mixed bed material sediment suspension; the evenly mixed bed material sediment suspension is obtained by evenly mixing the bed material sediment sample and distilled water or by evenly mixing the bed material sediment sample and salt-free water; and bed material sediment with different particle sizes in the bed material sediment sample settles to the preset plane at different settling rates;
a percent calculation module M2 configured to, for each of the particle sizes, calculate a percentage by weight of sediment finer than the particle size based on the intensity of light through the preset plane at each of the plurality of moments, where the percentage by weight of sediment finer than the particle size is a ratio of a sediment concentration of particle sizes smaller than the particle size to a total sediment concentration; and
a particle size analysis module M3 configured to obtain a particle size distribution and a particle size distribution curve based on each of the particle sizes and the percentage by weight of sediment finer than the particle size.

### Embodiment 3

In an exemplary embodiment, provided is a computer device, which may be a server or a terminal, and an internal structure thereof may be as shown in FIG. 5. The computer device includes a processor, a memory, an input/output (I/O) interface and a communication interface. The processor, the memory, and the input/output interface are connected via a system bus, and the communication interface is connected to the system bus via the input/output interface. The processor of the computer device is configured to provide computing and control capabilities. The memory of the computer device includes a nonvolatile storage medium and an internal memory. The non-volatile storage medium stores an operating system, a computer program, and a database. The internal memory provides an environment for operation of the operating system and the computer program in the nonvolatile storage medium. The database of the computer device is configured to store data. The input/output interface of the computer device is configured to exchange information between the processor and an external device. The communication interface of the computer device is configured to communicate with an external terminal through a network. The computer program, when executed by the processor, causes the method for particle size analysis of bed material sediment to be implemented.

Those skilled in the art may understand that the structure shown in FIG. 5 is only a block diagram of a part of the structure related to the solution of the present application and does not constitute a limitation on a computer device to which the solution of the present application is applied. Specifically, the computer device may include more or less components than those shown in the figure, or combine some components, or have different component arrangements.

In an exemplary embodiment, a computer device is provided, which includes a memory and a processor, where the memory has a computer program stored therein, and the computer program, when executed by the processor, causes the method for particle size analysis of bed material sediment in Embodiment 1 to be implemented.

### Embodiment 4

In an exemplary embodiment, a computer-readable storage medium is provided, which has a computer program stored therein. The computer program, when executed by a processor, causes the method for particle size analysis of bed material sediment in Embodiment 1 to be implemented.

### Embodiment 5

In an exemplary embodiment, a computer program product is provided, including a computer program. The computer program, when executed by a processor, causes the method for particle size analysis of bed material sediment in Embodiment 1 to be implemented.

It is to be noted that user information (including, but not limited to, user device information, user personal information, and the like) and data (including, but not limited to, data for analysis, data for storage, data for presentation, and the like) involved in the present application are information and data authorized by the user or fully authorized by each party, and relevant data shall be collected, used, and processed according to the related regulations.

The technical characteristics of the above embodiments can be employed in arbitrary combinations. To provide a concise description of these embodiments, all possible combinations of all the technical characteristics of the above embodiments may not be described. However, these combinations of the technical characteristics should be construed as falling within the scope defined by the specification as long as no contradiction occurs.

Several examples are used herein for illustration of the principles and implementations of the present application. The description of the foregoing examples is used to help illustrate the method of the present application and the core principles thereof. In addition, those of ordinary skill in the art can make various modifications in terms of specific implementations and scope of application in accordance with the teachings of the present application. In conclusion, the content of the present specification shall not be construed as a limitation to the present application.

What is claimed is:

1. A method for particle size analysis of bed material sediment, comprising:
obtaining an intensity of light through a preset plane at each of a plurality of moments during a settling process of a bed material sediment sample, wherein the preset plane is any horizontal plane other than a highest horizontal plane and a lowest horizontal plane in a sample container holding an evenly mixed bed material sediment suspension; the evenly mixed bed material sediment suspension is obtained by evenly mixing the bed material sediment sample and distilled water or by evenly mixing the bed material sediment sample and salt-free water; and bed material sediment with different particle sizes in the bed material sediment sample settles to the preset plane at different settling rates;
for any one of the particle sizes, calculating a percentage by weight of sediment finer than the any one of the particle sizes based on the intensity of light through the preset plane at each of the plurality of moments, wherein the percentage by weight of sediment finer than the any one of the particle sizes is a ratio of a sediment concentration of particle sizes smaller than the any one of the particle sizes to a total sediment concentration; and
obtaining a particle size distribution and a particle size distribution curve based on the any one of the particle sizes and the percentage by weight of sediment finer than the any one of the particle sizes.

2. The method for particle size analysis of bed material sediment according to claim 1, wherein a formula for calculating the percentage by weight of sediment finer than the any one of the particle sizes is as follows:
$${P}_{i} = \frac{{\sum }_{i = i}^{n - 1} \frac{2.3 {D}_{i , i + 1}}{kL} ( lg \frac{{I}_{0}}{{I}_{{t}_{i}}} - lg \frac{{I}_{0}}{{I}_{{t}_{i + 1}}} )}{{\sum }_{i = 1}^{n - 1} \frac{2.3 {D}_{i , i + 1}}{kL} ( lg \frac{{I}_{0}}{{I}_{{t}_{i}}} - lg \frac{{I}_{0}}{{I}_{{t}_{i + 1}}} )}$$
wherein Pᵢ represents a percentage by weight of sediment finer than an i-th particle size; n represents a number of particle sizes; D_{i, i+1} represents a particle size between the i-th particle size and an (i+1)-th particle size; k represents an extinction coefficient; L represents a thickness of the evenly mixed bed material sediment suspension; I₀ represents a light intensity at a 0-th moment; Iₜ_{ᵢ} represents a light intensity at a moment tᵢ when the bed material sediment of the i-th particle size settles to the preset plane; and Iₜ_{ᵢ₊₁} represents a light intensity at a moment tᵢ₊₁ when the bed material sediment of the (i+1)-th particle size settles to the preset plane.

3. The method for particle size analysis of bed material sediment according to claim 1, wherein when the method for particle size analysis of bed material sediment is applied to suspended load measurement or bed load measurement, the bed material sediment sample is a sample obtained after performing water sample treatment on a bed material sediment water sample; and the bed material sediment water sample is a bed material sediment water sample of each layer obtained by performing stratified sampling at each bed material sampling vertical in each measurement run;
the water sample treatment comprises filtering and drying the bed material sediment water sample to obtain the bed material sediment sample; and
during stratified sampling, a formula for calculating a sampling volume of the bed material sediment water sample is as follows:
$$V = \frac{{W}_{min}}{C}$$
wherein V represents the sampling volume of the bed material sediment water sample; Wₘᵢₙ represents a minimum sediment weight; and C represents a sediment concentration.

4. The method for particle size analysis of bed material sediment according to claim 1, wherein the sample container further holds a deflocculant.

5. The method for particle size analysis of bed material sediment according to claim 3, further comprising: calculating a vertical average particle size distribution at each bed material sampling vertical;
wherein a formula for calculating the vertical average particle size distribution is as follows:
$${\overline{P}}_{v} ( d ) = \frac{1}{H} {\sum }_{k = 1}^{m} {P}_{k} ( d ) \cdot Δ {h}_{k}$$
wherein P̅ᵥ(d) represents the vertical average particle size distribution; d represents a particle size; H represents a vertical depth; m represents a number of layers for stratified sampling; Pₖ(d) represents a particle size distribution at a k-th layer; and Δhₖ represents a sampling thickness of the k-th layer.

6. The method for particle size analysis of bed material sediment according to claim 5, further comprising: calculating a cross-sectional average particle size distribution and a cross-sectional average particle size of a cross section composed of all bed material sampling verticals at each measurement run;
wherein a formula for calculating the cross-sectional average particle size distribution is as follows:
$$\overline{{P}_{s}} ( d ) = \frac{{\sum }_{i = 1}^{X} {W}_{i} \overline{{P}_{{v}_{ι}}} ( d )}{{\sum }_{i = 1}^{X} {W}_{i}}$$
wherein P̅ₛ(d) represents the cross-sectional average particle size distribution; X represents a number of bed material sampling verticals; Wᵢ represents a sediment weight per unit width at an i-th bed material sampling vertical; and P̅ᵥ̅_̅{̅ₜ̅}̅(d) represents the vertical average particle size distribution at the i-th bed material sampling vertical;
$${W}_{i} = {\int }_{0}^{D} {ρ}_{s} \cdot {A}_{i} ( d ) \cdot d ( d )$$
wherein D represents a maximum particle size; ρₛ represents a bed material sediment density; and Aᵢ(d) represents a particle size distribution function for the i-th bed material sampling vertical; and
a formula for calculating the cross-sectional average particle size is as follows:
$${D}_{avg} = exp ( {\int }_{{d}_{min}}^{{d}_{max}} ln d \cdot d \overline{{P}_{s}} ( d ) )$$
wherein D_{avg} represents the cross-sectional average particle size; dₘₐₓ represents a maximum particle size value; and dₘᵢₙ represents a minimum maximum particle size value.

7. A device for particle size analysis of bed material sediment, comprising:
a light intensity obtaining module configured to obtain an intensity of light through a preset plane at each of a plurality of moments during a settling process of a bed material sediment sample, wherein the preset plane is any horizontal plane other than a highest horizontal plane and a lowest horizontal plane in a sample container holding an evenly mixed bed material sediment suspension; the evenly mixed bed material sediment suspension is obtained by evenly mixing the bed material sediment sample and distilled water or by evenly mixing the bed material sediment sample and salt-free water; and bed material sediment with different particle sizes in the bed material sediment sample settles to the preset plane at different settling rates;
a percent calculation module configured to, for any one of the particle sizes, calculate a percentage by weight of sediment finer than the any one of the particle sizes based on the intensity of light through the preset plane at each of the plurality of moments, wherein the percentage by weight of sediment finer than the any one of the particle sizes is a ratio of a sediment concentration of particle sizes smaller than the any one of the particle sizes to a total sediment concentration; and
a particle size analysis module configured to obtain a particle size distribution and a particle size distribution curve based on the any one of the particle sizes and the percentage by weight of sediment finer than the any one of the particle sizes.

8. A computer device, comprising a memory, a processor, and a computer program stored on the memory and runnable on the processor, wherein the processor is configured to execute the computer program to implement the method for particle size analysis of bed material sediment according to claim 1.

9. A non-transitory computer-readable storage medium, having a computer program stored therein, wherein the non-transitory computer program, when executed by a processor, causes the method for particle size analysis of bed material sediment according to claim 1 to be implemented.

10. The computer device according to claim 8, wherein a formula for calculating the percentage by weight of sediment finer than the any one of the particle sizes is as follows:
$${P}_{i} = \frac{{\sum }_{i = i}^{n - 1} \frac{2.3 {D}_{i , i + 1}}{kL} ( lg \frac{{I}_{0}}{{I}_{{t}_{i}}} - lg \frac{{I}_{0}}{{I}_{{t}_{i + 1}}} )}{{\sum }_{i = i}^{n - 1} \frac{2.3 {D}_{i , i + 1}}{kL} ( lg \frac{{I}_{0}}{{I}_{{t}_{i}}} - lg \frac{{I}_{0}}{{I}_{{t}_{i + 1}}} )}$$
wherein Pᵢ represents a percentage by weight of sediment finer than an i-th particle size; n represents a number of particle sizes; D_{i, i+1} represents a particle size between the i-th particle size and an (i+1)-th particle size; k represents an extinction coefficient; L represents a thickness of the evenly mixed bed material sediment suspension; I₀ represents a light intensity at a 0-th moment; Iₜ_{ᵢ} represents a light intensity at a moment tᵢ when the bed material sediment of the i-th particle size settles to the preset plane; and Iₜ_{ᵢ₊₁} represents a light intensity at a moment tᵢ₊₁ when the bed material sediment of the (i+1)-th particle size settles to the preset plane.

11. The computer device according to claim 8, wherein when the method for particle size analysis of bed material sediment is applied to suspended load measurement or bed load measurement, the bed material sediment sample is a sample obtained after performing water sample treatment on a bed material sediment water sample; and the bed material sediment water sample is a bed material sediment water sample of each layer obtained by performing stratified sampling at each bed material sampling vertical in each measurement run;
the water sample treatment comprises filtering and drying the bed material sediment water sample to obtain the bed material sediment sample; and
during stratified sampling, a formula for calculating a sampling volume of the bed material sediment water sample is as follows:
$$V = \frac{{W}_{min}}{C}$$
wherein V represents the sampling volume of the bed material sediment water sample; Wₘᵢₙ represents a minimum sediment weight; and C represents a sediment concentration.

12. The computer device according to claim 8, wherein the sample container further holds a deflocculant.

13. The computer device according to claim 11, further comprising: calculating a vertical average particle size distribution at each bed material sampling vertical;
wherein a formula for calculating the vertical average particle size distribution is as follows:
$${\overline{P}}_{v} ( d ) = \frac{1}{H} {\sum }_{k = 1}^{m} {P}_{k} ( d ) \cdot Δ {h}_{k}$$
wherein P̅ᵥ(d) represents the vertical average particle size distribution; d represents a particle size; H represents a vertical depth; m represents a number of layers for stratified sampling; Pₖ(d) represents a particle size distribution at a k-th layer; and Δhₖ represents a sampling thickness of the k-th layer.

14. The computer device according to claim 13, further comprising: calculating a cross-sectional average particle size distribution and a cross-sectional average particle size of a cross section composed of all bed material sampling verticals at each measurement run;
wherein a formula for calculating the cross-sectional average particle size distribution is as follows:
$$\overline{{P}_{s}} ( d ) = \frac{{\sum }_{i = 1}^{X} {W}_{i} \overline{{P}_{{v}_{ι}}} ( d )}{{\sum }_{i = 1}^{X} {W}_{i}}$$
wherein P̅ₛ(d) represents the cross-sectional average particle size distribution; X represents a number of bed material sampling verticals; Wᵢ represents a sediment weight per unit width at an i-th bed material sampling vertical; and P̅ᵥ̅_̅{̅ₜ̅}̅(d) represents the vertical average particle size distribution at the i-th bed material sampling vertical;
$${W}_{i} = {\int }_{0}^{D} {ρ}_{s} \cdot {A}_{i} ( d ) \cdot d ( d )$$
wherein D represents a maximum particle size; ρₛ represents a bed material sediment density; and Aᵢ(d) represents a particle size distribution function for the i-th bed material sampling vertical; and
a formula for calculating the cross-sectional average particle size is as follows:
$${D}_{avg} = exp ( {\int }_{{d}_{min}}^{{d}_{max}} ln d \cdot d \overline{{P}_{s}} ( d ) )$$
wherein D_{avg} represents the cross-sectional average particle size; dₘₐₓ represents a maximum particle size value; and dₘᵢₙ represents a minimum maximum particle size value.

15. The non-transitory computer-readable storage medium according to claim 9, wherein a formula for calculating the percentage by weight of sediment finer than the any one of the particle sizes is as follows:
$${P}_{i} = \frac{{\sum }_{i = i}^{n - 1} \frac{2.3 {D}_{i , i + 1}}{kL} ( lg \frac{{I}_{0}}{{I}_{{t}_{i}}} - lg \frac{{I}_{0}}{{I}_{{t}_{i + 1}}} )}{{\sum }_{i = i}^{n - 1} \frac{2.3 {D}_{i , i + 1}}{kL} ( lg \frac{{I}_{0}}{{I}_{{t}_{i}}} - lg \frac{{I}_{0}}{{I}_{{t}_{i + 1}}} )}$$
wherein Pᵢ represents a percentage by weight of sediment finer than an i-th particle size; n represents a number of particle sizes; D_{i, i+1} represents a particle size between the i-th particle size and an (i+1)-th particle size; k represents an extinction coefficient; L represents a thickness of the evenly mixed bed material sediment suspension; I₀ represents a light intensity at a 0-th moment; Iₜ_{ᵢ} represents a light intensity at a moment tᵢ when the bed material sediment of the i-th particle size settles to the preset plane; and Iₜ_{ᵢ₊₁} represents a light intensity at a moment tᵢ₊₁ when the bed material sediment of the (i+1)-th particle size settles to the preset plane.

16. The non-transitory computer-readable storage medium according to claim 9, wherein when the method for particle size analysis of bed material sediment is applied to suspended load measurement or bed load measurement, the bed material sediment sample is a sample obtained after performing water sample treatment on a bed material sediment water sample; and the bed material sediment water sample is a bed material sediment water sample of each layer obtained by performing stratified sampling at each bed material sampling vertical in each measurement run;
the water sample treatment comprises filtering and drying the bed material sediment water sample to obtain the bed material sediment sample; and
during stratified sampling, a formula for calculating a sampling volume of the bed material sediment water sample is as follows:
$$V = \frac{{W}_{min}}{C}$$
wherein V represents the sampling volume of the bed material sediment water sample; Wₘᵢₙ represents a minimum sediment weight; and C represents a sediment concentration.

17. The non-transitory computer-readable storage medium according to claim 9, wherein the sample container further holds a deflocculant.

18. The non-transitory computer-readable storage medium according to claim 16, further comprising: calculating a vertical average particle size distribution at each bed material sampling vertical;
wherein a formula for calculating the vertical average particle size distribution is as follows:
$${\overline{P}}_{v} ( d ) = \frac{1}{H} {\sum }_{k = 1}^{m} {P}_{k} ( d ) \cdot Δ {h}_{k}$$
wherein P̅ᵥ(d) represents the vertical average particle size distribution; d represents a particle size; H represents a vertical depth; m represents a number of layers for stratified sampling; Pₖ(d) represents a particle size distribution at a k-th layer; and Δhₖ represents a sampling thickness of the k-th layer.

19. The non-transitory computer-readable storage medium according to claim 18, further comprising: calculating a cross-sectional average particle size distribution and a cross-sectional average particle size of a cross section composed of all bed material sampling verticals at each measurement run;
wherein a formula for calculating the cross-sectional average particle size distribution is as follows:
$$\overline{{P}_{s}} ( d ) = \frac{{\sum }_{i = 1}^{X} {W}_{i} \overline{{P}_{{v}_{ι}}} ( d )}{{\sum }_{i = 1}^{X} {W}_{i}}$$
wherein P̅ₛ(d) represents the cross-sectional average particle size distribution; X represents a number of bed material sampling verticals; Wᵢ represents a sediment weight per unit width at an i-th bed material sampling vertical; and P̅ᵥ̅_̅{̅ₜ̅}̅(d) represents the vertical average particle size distribution at the i-th bed material sampling vertical;
$${W}_{i} = {\int }_{0}^{D} {ρ}_{s} \cdot {A}_{i} ( d ) \cdot d ( d )$$
wherein D represents a maximum particle size; ρₛ represents a bed material sediment density; and Aᵢ(d) represents a particle size distribution function for the i-th bed material sampling vertical; and
a formula for calculating the cross-sectional average particle size is as follows:
$${D}_{avg} = exp ( {\int }_{{d}_{min}}^{{d}_{max}} ln d \cdot d \overline{{P}_{s}} ( d ) )$$
wherein D_{avg} represents the cross-sectional average particle size; d_{wax} represents a maximum particle size value; and dₘᵢₙ represents a minimum maximum particle size value.
